(19) United States
(12) Patent Application Publication
(10) Pub. No.: US 2015/0132231 A1
(43) Pub. Date: May 14, 2015

(54) SURFACE FUNCTIONALIZATION OF SUPERPARAMAGNETIC NANOPARTICLES FOR BIOLOGICAL ENCAPSIDATION

(71) Applicant: The Board of Trustees of the Leland Stanford Junior University, Palo Alto, CA (US)

(72) Inventors: Benjamin Yen-Bin Ko, Menlo Park, CA (US); James Robert Swartz, Menlo Park, CA (US)

(21) Appl. No.: 14/533,783

(22) Filed: Nov. 5, 2014

Related U.S. Application Data
(60) Provisional application No. 61/901,967, filed on Nov. 8, 2013.

(51) Int. Cl. A61K 49/18; C12N 7/00

(52) U.S. Cl. CPC A61K 49/1836; A61K 49/1866; A61K 49/1896; C12N 7/00; B82Y 5/00
USPC 424/9.322; 424/9.32

(57) ABSTRACT

Method and compositions are provided that relate to superparamagnetic nanoparticles, including IONP with small diameter and high transverse relaxivity, that are stable in an aqueous environment. The nanoparticle surface coating is functionalized, while minimizing its thickness, to facilitate packaging within biological constructs.

### GOVERNMENT RIGHTS

This invention was made with Government support under contract W81XWH-10-1-0576 awarded by the Department of Defense. The Government has certain rights in the invention.

### BACKGROUND OF THE INVENTION

Recent advances in nanotechnology and biology include the development and application of functional nanoparticles that are covalently linked to biological molecules such as peptides, proteins, and nucleic acids or are enclosed within biomolecular assemblies. Due to their dimensional similarities to biomolecules and biomolecular assemblies, these nanoparticles are well suited as contrast agents for in vivo magnetic resonance imaging (MRI).

Superparamagnetic iron oxide nanoparticles (IONP) are of particular interest as contrast agents in imaging applications such as magnetic resonance imaging (MRI). IONP are available in various sizes with diameters on the order of 5 to 50 nm, which are suitable for a number of purposes, including packaging inside virus-like particles (VLP) or other protein constructs for the purpose of directing the IONP to a target of interest.

An IONP's MRI r2 (transverse) relaxivity, the key magnetic property related to its effectiveness as a contrast agent, is believed to be proportional to its volume. IONP with larger diameters are therefore more desirable payloads for imaging applications.

A variety of coating strategies done both in situ and post-synthesis have been developed for stabilization of magnetic nanoparticles to avoid their aggregation in aqueous environments. Post-synthesis coating processes include monolayer ligands, polymer coatings, and silica coatings. The monolayer ligand coatings rely on interactions of chemical groups from both the ligand and nanoparticle for effective adsorption or chemisorption of the ligand on the surface. These coatings tend to have limited colloidal stability due to weak steric hindrance in preventing aggregation and have limited opportunity for functionalization. Residual surfactant on the surface of nanoparticles can result in inefficient or incomplete coatings with this approach. The amphiphilic polymer-based or silica-based coating approach often results in multilayer coatings, making the coating process difficult to control and resulting in a heterogeneous sample, and sometimes in the encapsulation of dimers or trimers of nanoparticles in the same shell. In addition, many of these coating significantly increase the diameter of smaller IONPs and significantly reduce their transverse relaxivities.

Commercially produced IONP are currently available either with a thin oleic acid coating (believed a monolayer of about 2 nm), or with a thick polymeric coating (≧6 nm) that prevents nanoparticle aggregation in aqueous solutions. However, the oleic acid coating does not allow for IONP suspension in aqueous solutions, and the thickness of the polymeric coating severely limits the size of IONP that can be encapsidated.

The present invention provides compositions that overcome these issues.

U.S. Pat. No. 4,795,698 to Owen et al. relates to polymer-coated, colloidal, superparamagnetic particles which are produced by the formation of magnetite from Fe⁺²/Fe⁺³ salts in the presence of polymer. U.S. Pat. No. 4,452,773 to Molday describes a material similar in properties to those described in Owen et al., which is produced by forming magnetite and other iron oxides from Fe⁺²/Fe⁺³ via base addition in the presence of very high concentrations of dextran.

Another method for producing superparamagnetic, colloidal particles is described in U.S. Pat. No. 5,597,531. In contrast to the particles described in the Owen et al., or Molday patents, these latter particles are produced by directly coating a biofunctional polymer onto pre-formed superparamagnetic crystals which have been dispersed by high power sonic energy into quasi-stable crystalline clusters ranging from 25 to 120 nm.

### SUMMARY OF THE INVENTION

Method and compositions are provided that relate to superparamagnetic nanoparticles, including IONP, that are stable in an aqueous environment. The nanoparticle surface coating is functionalized, while minimizing its thickness, to facilitate packaging within biological constructs. In some embodiments, stable aqueous compositions of paramagnetic nanoparticles with small diameter and high transverse relaxivity are provided. In other embodiments, compositions are provided of encapsidated paramagnetic nanoparticles, which encapsidated paramagnetic nanoparticles may be used, for example, in imaging applications. In other embodiments, methods are provided for the functionalization of IONP to achieve a stable aqueous suspension of small diameter and high transverse relaxivity IONP.

In the methods of the invention, an oleic acid-coated superparamagnetic nanoparticle, e.g. a commercially available IONP, is surface functionalized during transfer from an organic phase to an aqueous phase. The oleic acid-coated IONP are suspended along with an amphipathic molecule in an organic solvent, e.g. chloroform, methylene chloride, methyl chloroform, tetrahydrofuran, xylol, halothane, desflurane, etc., preferably chloroform, and an aqueous layer (water or buffer) is then added. This mixture is sonicated to expand the interfacial surface area. Following sonication, the resultant emulsion is re-separated into its component layers via centrifugation. The aqueous layer then contains the functionalized IONP. In some embodiments, the amphipathic molecule is oleylamine.

In some embodiments, a stable aqueous suspension of functionalized IONP is provided, where the total diameter of the nanoparticle including coating is from about 1 nm to about 50 nm, from about 2 nm to about 30 nm, and may be from about 5 nm to about 25 nm. The transverse relaxivity R₂ of the nanoparticles is at least about 150 mM⁻¹_{Fe}s⁻¹, at least about 200 mM⁻¹_{Fe}s⁻¹, and may be 225 mM⁻¹_{Fe}s⁻¹ or more.

In some embodiments a composition is provided of a biological entity, including without limitation a virus-like particle (VLP), in which a functionalized IONP of the invention is encapsidated. In some embodiments the biological entity comprises a specific binding moiety for a target of interest. The biological entity may be formulated in a physiologically acceptable excipient.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Diagram of novel biphasic protocol for surface functionalization and phase transfer of IONP.

FIG. 2: Model for the process of converting the SPIO NPs from being hydrophobic (suspended in chloroform) to being hydrophilic. The dark squiggles indicate oleic acid and the light colored molecules indicate oleylamine. The red sphere is the 15 nm diameter SPIO NP, and the red box highlights the interfacial layer.

FIG. 3: Effect of intercalating agent concentration in organic layer on (a) phase transfer efficiency, and (b) transverse relaxivity.

FIG. 4: Zeta potential as a function of oleylamine to oleic acid ratio in the organic layer, at three different aqueous buffer pH values

FIG. 5: (a) Oleic acid-coated IONP before (l) and after (r) surface functionalization and water solubilization; (b) TEM image of water-soluble IONP (JEOL JEM-1400, 1% uranyl acetate-stained).

### DETAILED DESCRIPTION OF THE EMBODIMENTS

### Definitions

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

“Measuring” or “measurement” in the context of the present teachings refers to determining the presence, absence, quantity, amount, or effective amount of a substance in a clinical or subject-derived sample, including the presence, absence, or concentration levels of such substances, and/or evaluating the values or categorization of a subject's clinical parameters based on a control. Measuring emission of light, either integrated total emission or detection of particles corresponding to rare cells or biological entities may be performed.

Unless otherwise apparent from the context, all elements, steps or features of the invention can be used in any combination with other elements, steps or features.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Due to biological functional equivalency considerations, changes can be made in protein structure without affecting the biological action in kind or amount. All such modifications are intended to be included within the scope of the appended claims.

A magnetic nanoparticle comprises a metal selected from the group consisting of iron, cobalt, zinc, cadmium, nickel, gadolinium, chromium, copper, manganese, and their oxides. In other embodiments, the magnetic nanoparticle is an alloy with a metal selected from the group consisting of gold, silver, platinum, and copper. The invention further provides that the magnetic nanoparticle may comprise a free metal ion, a metal oxide, a chelate, or an insoluble metal compound. In certain embodiments, the magnetic nanoparticle is selected from the group consisting of Fe₃O₄, Fe₂O₄, FexPty, CoxPty, MnFexOy, CoFexoy, NiFexoy, CuFexoy, ZnFexOy, and CdFexOy, wherein x and y vary depending on the method of synthesis. In other embodiments, the magnetic nanoparticle further comprises a metal coating selected from the group consisting of gold, silver, iron, cobalt, zinc, cadmium, nickel, gadolinium, chromium, copper, and manganese, and an alloy thereof. In yet other embodiments, the magnetic nanoparticle comprises terbium or europium. In certain preferred embodiments, the magnetic nanoparticle is selected from the group consisting of monocrystalline iron oxide nanoparticle (MION), chelate of gadolinium, and superparamagnetic iron oxide (IONP). In a preferred embodiment, the magnetic nanoparticle is IONP.

The nanoparticles of the invention, including functionalization, typically have a diameter, for example as determined by DLS, of about 15 nm to 100 nm and in some embodiments between 5 and 50 nm, and in still other embodiments, between about 5 nm and about 25 nm. If the nanoparticles are destined for in vivo use as, for instance, targeted, VLP-enclosed MRI contrast agents, a particularly convenient diameter is about 30 nm or less.

Stable monodisperse aqueous colloidal suspensions of the functionalized nanoparticles are readily obtained. Such suspensions are preferably stable against filtration such as tangential flow filtration against a 30 kDa cut off membrane in aqueous solutions, preferably with low ionic strength. Additionally, the encapsidated IONPs should be stable after the addition of electrolytes such as the addition of NaCl to render the aqueous medium isotonic, i.e. about 150 mM of NaCl. Preferably the suspensions are stable for storage periods of at least one day, one week or greater and more preferably are stable against not only sedimentation but also against aggregation of the suspended nanoparticles. For example, a stable suspension may lose less than 5% of the nanoparticles to the pellet after the suspension has been stored for 24 hours. These suspensions should also not produce dimeric or polymeric nanoparticles that would prevent their encapsidation. After encapsidation, if the suspensions are intended for in vivo use in human subjects, it is convenient to render them isotonic by the addition of NaCl, dextrose, or with other tonicity modifiers known in the art or combinations thereof.

The functionalized nanoparticles of the invention may be conveniently used as therapeutic agents or as contrast agents in diagnostic imaging. Common types of diagnostic imaging include magnetic resonance (MR) imaging. A convenient approach to the administration of the nanoparticles to human subjects is to encapsidate the nanoparticles, and administer the resulting biological entity intraveneously, preferably as a stable isotonic aqueous suspension.

Amphipathic molecules suitable for functionalizing nanoparticles have a hydrophilic head group and a hydrophobic tail group, where the hydrophobic group and hydrophilic group are joined by a covalent bond, or by a variable length linker group. The linker portion may be a bifunctional aliphatic compound which can include heteroatoms or bifunctional aromatic compounds. Amphipathic molecules of interest include fatty acids and fatty acid esters, preferably with aliphatic side chains of comparable length to that used for the initial hydrophobic coating. Other acceptable amphipathic molecules can readily be identified using methods taught be this invention. Specific amphipathic molecules of interest include, without limitation, long-chain aliphatic amines, e.g. hexadecylamine, octylamine, and oleylamine, phospholipids, e.g. DOPE and DPPE; and fatty acids, e.g. oleic acid.

As used herein, the term “virus like particle” refers to a stable macromolecular assembly of one or more virus proteins, usually viral coat proteins. The number of separate protein chains in a VLP will usually be at least about 60 proteins, about 80 proteins, at least about 120 proteins, or more, depending on the specific viral geometry. The capsid may be empty, or contain non-viral components, e.g. mRNA fragments, etc. A preferred virus protein for assembly into a VLP is Hepatitis B core protein, although other virus proteins also find use for this purpose.

The VLP may comprise a specific binding moiety, e.g. for targeting to a tissue, tumor, etc. The binding moiety may be directly conjugated to the VLP through covalent bonds. Conveniently, an unnatural amino acid and CLICK chemistry is used to make a stable labeling nanoparticle for these purposes. An advantage of the methods of the invention is the ability to generate a nanoparticle with multiple labeling and affinity moieties on a single nanoparticle, e.g. at least about 5 conjugated moieties, at least about 10 conjugated moieties, at least about 20 conjugated moieties, at least about 30 conjugated moieties, at least about 40 conjugated moieties, at least about 60 conjugated moieties, at least about 80 conjugated moieties, where the moieties may be the same or different.

A stable labeling nanoparticle maintains the association of proteins in a capsid structure under physiological conditions for extended periods of time, e.g. for at least about 24 hrs, at least about 1 week, at least about 1 month, or more. Once assembled, the nanoparticles can have a stability commensurate with the native virus particle and, in some cases, exceed that stability, e.g. upon exposure to pH changes, heat, freezing, ionic changes, etc.

The term “polypeptide,” “peptide,” “oligopeptide,” and “protein,” are used interchangeably herein, and refer to a polymeric form of amino acids of any length, which can include coded and non-coded amino acids, chemically, or biochemically modified or derivatized amino acids, and polypeptides having modified peptide backbones.

As used herein, the terms “purified” and “isolated” when used in the context of a polypeptide that is substantially free of contaminating materials from the material from which it was obtained, e.g. cellular materials, such as but not limited to cell debris, cell wall materials, membranes, organelles, the bulk of the nucleic acids, carbohydrates, proteins, and/or lipids present in cells. Thus, a polypeptide that is isolated includes preparations of a polypeptide having less than about 30%, 20%, 10%, 5%, 2%, or 1% (by dry weight) of cellular materials and/or contaminating materials. As used herein, the terms “purified” and “isolated” when used in the context of a polypeptide that is chemically synthesized refers to a polypeptide which is substantially free of chemical precursors or other chemicals which are involved in the syntheses of the polypeptide.

Polypeptides may be isolated and purified in accordance with conventional methods of recombinant synthesis or cell free protein synthesis. Methods which are well known to those skilled in the art can be used to construct expression vectors containing coding sequences and appropriate transcriptional/translational control signals. These methods include, for example, in vitro recombinant DNA techniques, synthetic techniques and in vivo recombination/genetic recombination. Alternatively, RNA capable of encoding the polypeptides of interest may be chemically synthesized. One of skill in the art can readily utilize well-known codon usage tables and synthetic methods to provide a suitable coding sequence for any of the polypeptides of the invention. The nucleic acids may be isolated and obtained in substantial purity. Usually, the nucleic acids, either as DNA or RNA, will be obtained substantially free of other naturally-occurring nucleic acid sequences, generally being at least about 50%, usually at least about 90% pure and are typically “recombinant,” e.g., flanked by one or more nucleotides with which it is not normally associated on a naturally occurring chromosome. The nucleic acids of the invention can be provided as a linear molecule or within a circular molecule, and can be provided within autonomously replicating molecules (vectors) or within molecules without replication sequences. Expression of the nucleic acids can be regulated by their own or by other regulatory sequences known in the art. The nucleic acids of the invention can be introduced into suitable host cells using a variety of techniques available in the art.

Viral proteins for generating VLP may comprise one or more unnatural amino acids at a pre-determined site, and may comprise or contain 1, 2, 3, 4, 5 or more unnatural amino acids. If present at two or more sites in the polypeptide, the unnatural amino acids can be the same or different.

Unnatural amino acids of interest include, without limitation, amino acids that provide a reactant group for CLICK chemistry reactions (see Click Chemistry: Diverse Chemical Function from a Few Good Reactions Hartmuth C. Kolb, M. G. Finn, K. Barry Sharpless Angewandte Chemie International Edition Volume 40, 2001, P. 2004, herein specifically incorporated by reference). For example, the amino acids azidohomoalanine, p-acetyl-L-phenylalanine and p-azido-L-phenylalanine are of interest. In some embodiments, the unnatural amino acid is introduced by global replacement of methionine on the protein, e.g. methionine can be left out of a cell-free reaction mixture, and substituted by from 0.25-2.5 mM azidohomoalanine (AHA). Alternatively the unnatural amino acid is introduced by orthogonal components, as known in the art.

Binding moieties may comprise a complementary active group for CLICK chemistry conjugation to the viral polypeptide of the invention. For example, it may be synthesized with one or more unnatural amino acids, which allow for the conjugation to the unnatural amino acid present on the viral protein. One of skill in the art will understand that the chemistry for conjugation is well-known and can be readily applied to a variety of groups, e.g. detectable label, antibody, polypeptide, etc.

The term “specific binding moiety” or “binding member” as used herein refers to a member of a specific binding pair, i.e. two molecules, usually two different molecules, where one of the molecules (i.e., first specific binding member) through chemical or physical means specifically binds to the other molecule (i.e., second specific binding member). The complementary members of a specific binding pair are sometimes referred to as a ligand and receptor; or receptor and counter-receptor. For the purposes of the present invention, the two binding members may be known to associate with each other, for example where an assay is directed at detecting compounds that interfere with the association of a known binding pair. Alternatively, candidate compounds suspected of being a binding partner to a compound of interest may be used.

Specific binding pairs of interest include carbohydrates and lectins; peptide ligands and receptor; effector and receptor molecules; hormones and hormone binding protein; enzyme cofactors and enzymes; enzyme inhibitors and enzymes; etc. The specific binding pairs may include analogs, derivatives and fragments of the original specific binding member. For example, a receptor and ligand pair may include peptide fragments, chemically synthesized peptidomimetics, labeled protein, derivatized protein, etc.

Compounds of interest as binding pair members encompass numerous chemical classes, though typically they are organic molecules. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Compounds are found among biomolecules including peptides, saccharides, fatty acids, steroids, purines, pyrimidines, derivatives, structural analogs or combinations thereof.

A binding moiety of interest includes an antibody. The term “antibody,” as used herein, refers to immunoglobulins that are produced in response to the detection of a foreign substance, and includes intact molecules as well as functional fragments thereof, such as Fab, F(ab′)₂, and Fv, single chain Fv, etc.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer. Subjects may be human, but also include other mammals, particularly those mammals useful as laboratory models for human disease, e.g. mouse, rat, etc. Also included are mammals such as domestic and other species of canines, felines, and the like.

### Compositions and Methods

A composition of oleic acid-coated nanoparticles, including without limitation IONP, are functionalized to improve aqueous stability while maintaining a desired size and transverse relaxivity. Oleic acid-coated nanoparticles are commercially available from different sources.

The oleic acid-coated IONP are suspended at a concentration of from about 0.1 mg Fe/mL, from about 1 mg Fe/mL, from about 5 mg Fe/mL up to about 500 mg Fe/mL, up to about 100 mg Fe/mL, up to about 50 mg Fe/mL in an organic solvent, including without limitation chloroform, methylene chloride, methyl chloroform, tetrahydrofuran, xylol, halothane, desflurane, etc., preferably chloroform, with an amphipathic molecule. Amphipathic molecules are added to the organic phase at a molar concentration of at least about 5 mM, at least about 10 mM, at least about 50 mM, at least about 100 mM, up to about 500 mM, up to about 250 mM in the organic phase. Amphipathic molecules of interest may be positively charged, negatively charged, or of neutral charge.

An aqueous layer is added, and the resulting suspension is sonicated to expand the interfacial surface area, for a period of at least about at least about 1 min., at least about 5 min. and not more than about 45 min., not more than about 30 min., and may be around about 15-20 min. The output frequency may be at least about 0.5 kHz, at least about 1 kHz, at least about 5 kHz, at least about 10 kHz and may be up to 20 kHz. The aqueous phase may be buffered, e.g. 10 mM Gly HCl, pH 3; 10 mM HEPES, pH 7; and 10 mM Tris base, pH 10; and may have a low ionicity, e.g. not more than about 100 mM NaCaI or the equivalent. The ratio of aqueous:organic layer volume ratio may range from 1:1, 2:1, 3:1, 5:1, 9:1, 10:1, 15:1; 1:2; 1:5 etc.

Following sonication, the resultant emulsion is re-separated into its component layers via centrifugation, for example at 20,000×g, for a period of from about 1 minute, from about 2 minutes, from about 5 minutes, up to about 30 minutes, up to about 20 minutes. The functionalized nanoparticles are suspended in the aqueous layer.

When used in diagnostic imaging, particularly of mammalian subjects and more particularly of human subjects, the functionalized nanoparticles of the invention, which may be encapsidated, are typically taken up in a pharmaceutically acceptable carrier which may or may not comprise one or more excipients. If the administration is to be by injection, particularly parenteral injection, the carrier is typically an aqueous medium that has been rendered isotonic by the addition of about 150 mM of NaCl, 5% dextrose or combinations thereof. It typically also has the physiological pH of between about 7.3 and 7.4. The administration may be intramuscular (IM), subcutaneous (SQ) or most commonly intravenous (IV). However, the administration may also be via implantation of a depot that then slowly releases the nanoparticles to the subject's blood or tissue.

The administration to human subjects, particularly IV administration, requires that the nanoparticles are non-toxic in the amounts used and free of any infective agents such as bacteria and viruses and also free of any pyrogens. Thus, these nanoparticles should be stable to the necessary purification procedures.

These nanoparticles may be delivered to the site of administration as a stable aqueous colloidal suspension with the proper osmolality and pH, as a concentrated aqueous colloidal suspension suitable for dilution and adjustment or as a powder, such as obtained by lyophilization, suitable for reconstitution.

The present disclosure, therefore, further relates to methods for imaging a subject using the nanoparticles disclosed herein. Such method comprises the step of subjecting a subject to whom a nanoparticle composition has been administered to imaging, for example by magnetic resonance, wherein said composition administered to the subject comprises nanoparticles of the invention, optionally encapsidated in a biological entity such as a VLP.

Also within the scope of the invention are kits comprising the compositions (e.g., the functionalized nanoparticles, encapsidated functionalized nanoparticles, and formulations thereof) of the invention and instructions for use. Kits typically include a label indicating the intended use of the contents of the kit. The term label includes any writing, or recorded material supplied on or with the kit, or which otherwise accompanies the kit.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the reagents, cells, constructs, and methodologies that are described in the publications, and which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### Example 1

### Surface Functionalization of Iron Oxide Nanoparticles for Biological Encapsidation

### Materials and Methods

Chemicals and reagents. Oleic acid-coated IONP with diameters of 5, 10, and 15 nm were obtained from Ocean NanoTech (Springdale, Ark.). Amphipathic ligands—oleylamine (CAS #112-90-3) and oleic acid (CAS #112-80-1)—were of the highest available quality and purchased from Sigma-Aldrich (St. Louis, Mo.).

Equipment. A Sonic Dismembrator 550 (Fisher Scientific) with a microtip probe was used to sonicate the organic and aqueous mixture. An Eppendorf Microcentrifuge 5418 was used to centrifuge the emulsion samples.

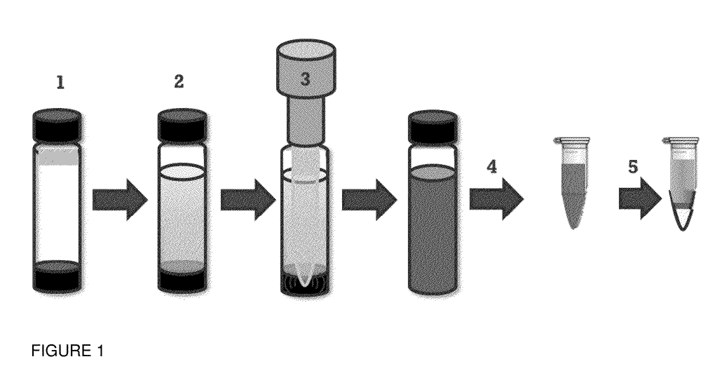

Phase transfer of IONP. A biphasic protocol was developed for transfer of IONP from organic to aqueous phase, with concurrent functionalization of the IONP surface (FIG. 1). First, oleic acid-coated IONP at an initial concentration of 5 mg/mL Fe and an amphipathic molecule, for example oleylamine or oleic acid, were mixed together and dissolved in a total volume of 1 mL chloroform in a Wheaton 3 dram glass vial, and the vial was placed into an ice bath. 9 mL of mQ H₂O or aqueous buffer (10 mM HEPES, pH 7) were overlayed on top of the chloroform layer. Next, the sonicator probe was placed into the glass vial, with the tip of the probe localized to the interface between the water and chloroform layers. Sonication of the water/chloroform mixture was performed for 15 minutes at 20 kHz with an intensity setting of 3.5. During this period of time, the vial was adjusted as necessary to ensure complete mixing and to create a homogeneous emulsion, and ice was replenished as necessary to maintain submersion of the vial. The resultant emulsion was then separated into ˜1 mL aliquots, such that the aliquots could be centrifuged in a microcentrifuge at 20,000×g for 5 minutes. The top colored (aqueous) layer of each aliquot was then combined into a single sample for subsequent analysis.

Analytical methods. The aqueous layer was analyzed for phase transfer efficiency, relaxivity, particle size distribution, and zeta potential.

For phase transfer efficiency, we determined the concentration of Fe in the aqueous layer and then performed a mass balance based on the initial mass of Fe in the IONP. Fe concentration was determined using either inductively coupled plasma with optical emission spectroscopy (ICP-OES) or mass spectrometry (ICP-MS), or a colorimetric assay in which the sample is incubated with 5 M hydrochloric acid at high temperature and the resultant A₂₆₁ is measured (Rad et al., BioTechniques, 2007).

Transverse relaxation times (T₂) were determined via ¹H NMR using an Inova 300 NMR (300 Hz, 7.0 T). Transverse relaxivities (r₂) were then calculated using the following equation, where T_{2,obs} is the transverse relaxation time of the sample and T_{2,H2O} is that of water:

$${R}_{2} [ Fe ] = \frac{1}{{T}_{2 , obs}} - \frac{1}{{T}_{2 , {H}_{2} O}}$$

Particle size distribution and zeta potential measurements were determined using a Malvern Zetasizer ZS. For particle size, a 50 μL sample was placed into a microcuvette and analyzed for mean particle diameter. For zeta potential, a 800 μL sample was placed into a folded capillary cell. Data was analyzed using Zetasizer software v6.12.

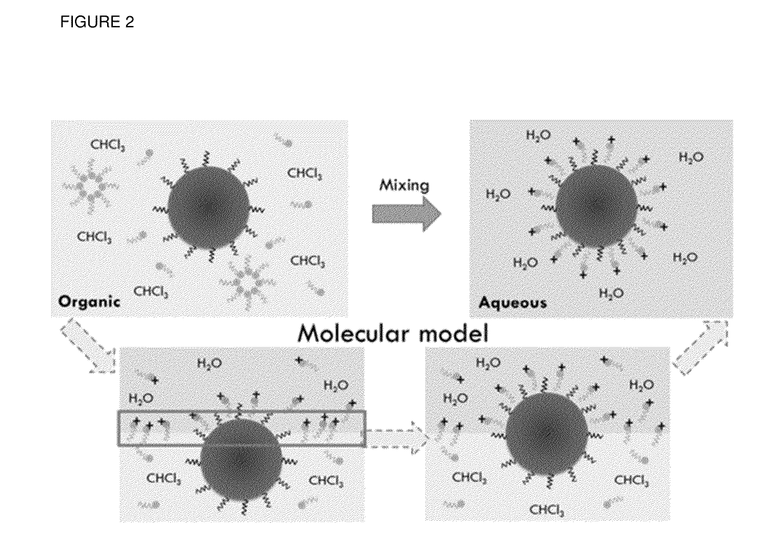

Here, we present a method for the surface functionalization of oleic acid-coated IONP during transfer from an organic phase to an aqueous phase. The oleic acid-coated IONP are first suspended along with an amphipathic molecule (e.g. oleylamine) in chloroform, and an aqueous layer (water or buffer) is then added. This mixture is sonicated to expand the chloroform/water interfacial surface area. Following sonication, the resultant emulsion is re-separated into its component layers via centrifugation, and the aqueous layer exhibits a dark-brown color, indicating successful phase transfer of the IONP. Our working hypothesis is that the amphipathic molecules can intercalate between the hydrophobic tails of the oleic acid molecules that are exposed on the surface of the IONP (the charged carboxylic acid group will associate with the surface of the IONP) (FIG. 2). At the chloroform/water interfacial surfaces, the hydrophobic end of the amphipathic oleylamine will intercalate into the IONP surface layer to add a positively charged amine onto the IONP surface. This will continue until the IONP is sufficiently hydrophilic to transition into the aqueous phase.

This procedure was tested with a variety of amphipathic molecules, and oleylamine was the optimal agent in terms of phase transfer efficiency. Various parameters were examined to determine the conditions for producing water-soluble IONP of desirable particle size and with the optimal combination of phase transfer efficiency and relaxivity. The data discussed below is for IONP with a core diameter of 15 nm, but similar results were produced for 5 and 10 nm diameter IONP.

Sonication time and intensity parameters were studied. At a sonicator intensity setting of 3.5, a sonication time of ≧15 minutes was determined to be necessary for maximizing the phase transfer efficiency and producing IONP of desired diameter (˜20 nm) (Table 1). The larger particle diameter at the 9 minute time point is possibly due to incomplete coating of the IONP with hydrophilic ligands, leading to partial aggregation. A higher sonicator intensity setting of 5.0 resulted in better phase transfer efficiency, but also a higher (and unacceptable) mean particle diameter. Additionally, the transverse relaxivity also decreased in this case. Therefore, a sonicator setting of 3.5 was deemed to be optimal.

TABLE 1
Effect of sonication time and intensity; [oleylamine]
in organic layer = 7.5 mM.

| | Sonication | Phase transfer | Transverse | Mean particle |
|---|---|---|---|---|
| Sonication | time | efficiency via | relaxivity | diameter via |
| intensity | (min) | [Fe] (%) | R₂ (mM⁻¹_{Fe}s⁻¹) | DLS (nm) |
| 3.5 | 5 | 11 | ND | ND |
| 3.5 | 9 | 20 | 241 | 32 |
| 3.5 | 15 | 21 | 226 | 18 |
| 5.0 | 15 | 59 | 149 | 49 |

ND = not determined

The effect of ionic strength was examined by varying the concentration of NaCl when using 10 mM glycine HCl, pH 2.5, as the aqueous phase buffer. As seen in Table 2, the mere presence of salt in the aqueous phase buffer resulted in IONP aggregation and markedly lower phase transfer efficiencies. Similar instances of IONP aggregation were seen when incubating the phase-transferred IONP in solutions containing other salts, such as magnesium glutamate.

TABLE 2
Effect of ionic strength; [oleylamine] in organic layer = 80 μM.

| [NaCl] in aqueous buffer | Phase transfer |
|---|---|
| (10 mM Gly HCl, pH 2.5) (mM) | efficiency via [Fe] (%) |
| 0 | 36 |
| 50 | 9 |
| 500 | <1 |

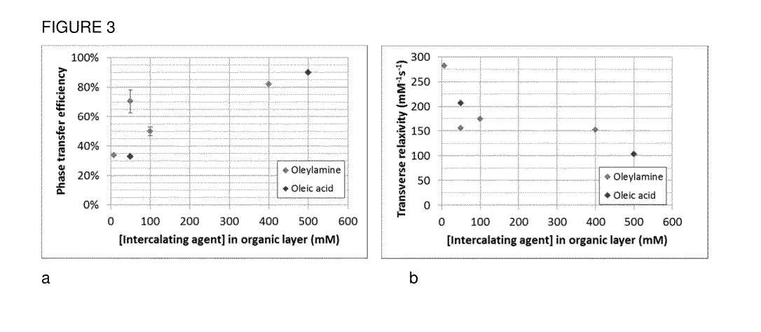

Although yields of hydrophilic IONP that were relatively low—about 20 to 30%, the newly converted hydrophilic IONPs had expected diameters (about 15 to 20 nm for 15 nm IONP) and also had very high transverse relaxivities (R2) when measured using NMR. Published values for IONP of comparable size range from 53 to 134 mM⁻¹_{Fe} s⁻¹ (Smolensky et al., Contrast Media Mol. Imaging, 2010), and we consistently observed R2 values 150 mM⁻¹_{Fe} s⁻¹. We next wanted to examine the effect of increasing the concentration of intercalating agent (amphipathic molecule) in the organic layer, expecting that an increase in the local concentration of intercalating agent around the aqueous/organic interface might result in improved phase transfer of IONP. In fact, this effect was observed (FIG. 3a). However, another consequence of higher intercalating agent concentration was a decrease in the transverse relaxivity of the IONP, as seen in FIG. 3b. Because the transverse relaxivity is such a key characteristic of a successful contrast agent, we decided that the optimal intercalating agent concentration for our application was ˜50 mM, where we could obtain R2 values ≧150 mM⁻¹_{Fe} s⁻¹ and still have phase transfer efficiencies of 30%.

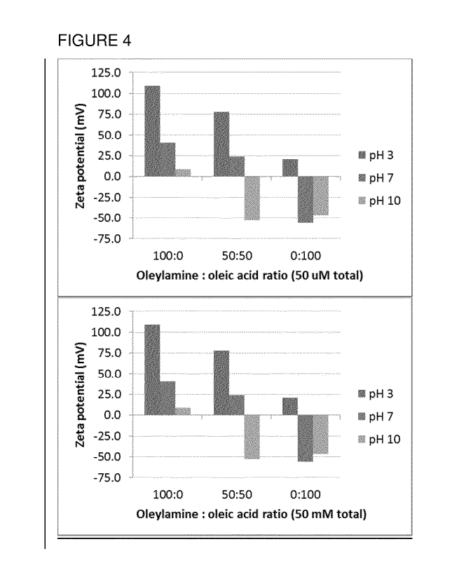

FIG. 3 also demonstrates that similar results are obtained when using a negatively charged intercalating agent (oleic acid) rather than one with a positive charge (oleylamine). Crucially, this allows us to functionalize the surface of the IONP with a desired charge. Potentially, this also indicates that we may be able to “tune” the surface charge characteristics of the IONP by varying the ratio of intercalating agents with (+) and (−) charges. Preliminary data demonstrates that the zeta potential, an indicator of surface charge, varies depending on the aqueous buffer pH and the ratio of oleylamine to oleic acid present in the organic layer (FIG. 4).

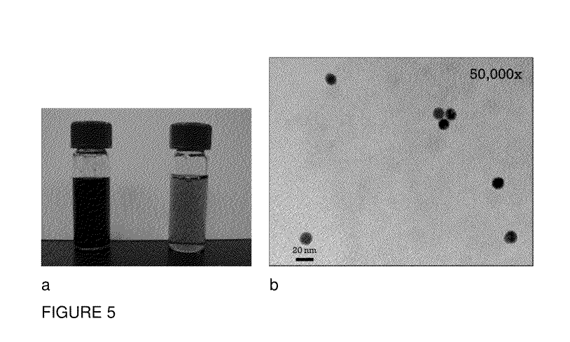

We have demonstrated that IONP produced using this surface functionalization method are water-soluble and generally monodisperse (FIG. 5). They have a number of advantages over currently available IONP. Firstly, our functionalized IONP have been rendered water-soluble, making them more suitable for biological applications than simple oleic acid-coated IONP. In comparison with existing water-soluble IONP, our IONP have a much thinner surface coating (2 to 3 nm, compared with ≧6 nm); thus, a larger IONP can be packaged inside a construct of particular size. Finally, IONP produced using the method described here have significantly higher relaxivities than comparable commercially-available IONP. We have consistently observed r2 values ≧150 mM⁻¹_{Fe} s⁻¹, compared with the range of 53 to 134 mM⁻¹_{Fe} s⁻¹ reported in literature. In fact, we measured the relaxivity of a commercial water-soluble IONP

The following examples are provided to facilitate the practice of the present invention. These examples are not intended to limit the scope of the invention in any way.

1. A method of generating functionalized superparamagnetic iron oxide nanoparticles (IONP) of small diameter and high transverse relaxivity, the method comprising:
suspending oleic-acid coated IONP in an organic solvent in the presence of an amphipathic molecule and an aqueous layer to generate an emulsion;
sonicating the emulsion;
centrifuging the sonicate to separate the layers;
collecting the aqueous layer and functionalized nanoparticles comprises therein.

2. The method of claim 1, wherein the organic solvent is chloroform.

3. The method of claim 1, wherein the amphipathic molecules is oleylamine.

4. The method of claim 1 wherein the functionalized nanoparticles are from 1 to 50 nm in diameter.

5. The method of claim 1, wherein the functionalized nanoparticles are from 2 to 30 nm in diameter.

6. The method of claim 1, wherein the transverse relaxivity R₂ of the functionalized nanoparticles is at least about 150 mM⁻¹_{Fe}S⁻¹.

7. The method of claim 1, wherein the transverse relaxivity R₂ of the functionalized nanoparticles is at least about 200 mM⁻¹_{Fe}S⁻¹.

8. A stable aqueous suspension of functionalized nanoparticles as produced by the method of claim 1.

9. A biological entity comprising functionalized nanoparticles as produced by the method of claim 1.

10. The biological entity of claim wherein the entity is a virus like particle.
